SEQUENCE LISTING (8 sequences)

SEQ ID NO: 1 - protein, 513 aa, Artificial Sequence
  RUNX2 amino acid sequence
MRIPVDPSTSRRFSPPSSSLQPGKMSDVSPVVAAQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 2 - DNA, 1542 bp, Artificial Sequence
  runx2 DNA sequence
atgcgtattcctgtagatccgagcaccagccggcgcttcagccccccctccagcagcctgcagcccggcaagatgagcgacgtgagcccggtggtggctgcgcagcagcagcaacagcagcagcagcaacagcagcagcaacaacagcaacagcaacaacagcagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattga

SEQ ID NO: 3 - DNA, 24 bp, Artificial Sequence
  endogenous RUNX2 primer
gctgttaggcctcgtattcctgta

SEQ ID NO: 4 - DNA, 24 bp, Artificial Sequence
  endogenous RUNX2 primer
ccgctcgagtcaatatggccgcca

SEQ ID NO: 5 - DNA, 24 bp, Artificial Sequence
  exogenous RUNX2 primer
agcaagcttgatttaggtgacact

SEQ ID NO: 6 - DNA, 27 bp, Artificial Sequence
  exogenous RUNX2 primer
ccgctcgagtcttggttcccggggacc

SEQ ID NO: 7 - DNA, 30 bp, Artificial Sequence
  OC gene RT-PCR primer
gtgaattcacctagcagacaccatgaggac

SEQ ID NO: 8 - DNA, 30 bp, Artificial Sequence
  OC gene RT-PCR primer
acggatccgtcttcaagccatactggtctg

(19) United States
(12) Patent Application Publication
(10) Pub. No.: US 2008/0219962 A1
(43) Pub. Date: Sep. 11, 2008

(54) Method to Enhance the Bone Formation Activity of Bmp by Runx2 Acetylation

(75) Inventors: Suk-Chul Bae, Chungcheongbukdo (KR); Hyun-Mo Ryoo, Seoul (KR)

Correspondence Address: LUCAS & MERCANTI, LLP, 475 PARK AVENUE SOUTH, 15TH FLOOR, NEW YORK, NY, 10016, US

(73) Assignee: BIORUNX CO., LTD., Chungbuk (KR)

(21) Appl. No.: 11/721,616

(22) PCT Filed: Dec. 14, 2005
(86) PCT No.: PCT/KR05/04307

§ 371 Date: Jun. 13, 2007

(30) Foreign Application Priority Data
Dec. 14, 2005 (KR) 10-2004-0105703

(51) Int. Cl. A61K 38/43; A61K 38/16; A61P 19/08; C12N 9/78

(52) U.S. Cl. 424/94.1; 514/12; 435/227

(57) ABSTRACT

The present invention relates to a method to enhance the activity of Runx2, a major target protein of Bone Mophogenetic Protein (BMP), by Runx2 acetylation, more precisely, a method to activate BMP-mediated bone formation pathway by protecting Runx2 from ubiquitination, indicating that Runx2 is protected from degradation by the increase of Runx2 acetylation making the protein more stable. The method to enhance Runx2 activity of the present invention can be utilized for the prevention and the treatment of bone disease such as osteoporosis, osteogenesis imperfecta, periodontal disease and fracture, by inducing bone formation by inhibiting Runx2 degradation.

### TECHNICAL FIELD

The present invention relates to a method to enhance the activity of BMP by increasing the activity of Runx2, a major target protein of BMP, by Runx2 acetylation. More precisely, the present invention relates to a method to activate the bone formation pathway of BMP by increasing stability of Runx2, for which degradation of Runx2 by ubiquitination is suppressed by Runx2 acetylation.

### BACKGROUND ART

Bone disease is caused by the decrease of bone density. Most representative bone diseases are osteoporosis, periodontal disease, which causes symptoms of teeth-coming out and fracture because of reduced bone density. Such bone disease is becoming one of serious social problems which lower the quality of life in particular in an aging society. Therefore, numbers of studies have been undergoing to treat bone disease successfully.

Bone tissue is composed of three representative cell types deeply involved in bone metabolism, which are osteoblasts, osteoclasts and osteocytes. The functions and amount of bone tissue are carefully regulated by the equilibrium between bone formation by osteoblasts and bone resorption by osteoclasts. Prevention and treatment of bone disease such as osteoporosis or periodontal disease have depended on suppression of bone resorption by osteoclasts and enhancement of bone formation by osteoblasts.

For example, a bone resorption inhibitor having an activity of suppressing the functions of osteoclasts such as calcitonin or bisphosphonate derivatives has been developed and clinically used so far. However, these inhibitors have problems of doubtful pharmaceutical effect and inefficient administration method and period, etc.

Remarkable progress has been made in the development of decoy receptor and antibody of RANKL (receptor activator of NF ligand) inhibiting osteoclast differentiation, a peptide compound inhibiting the adhesion of osteoclasts on the surface of bone through integrin, c-src tyrosine kinase inhibitor suppressing the function of bone resorption of osteoclasts, a vacuole ATPase inhibitor playing a role in degradation of hydroxyapatite, and cathepsin K inhibitor engaged in decomposition of organisms of bone tissue.

Numbers of studies have been focused on the development of an inhibitor of bone resorption by osteoclasts, but not on the development of an accelerant of bone formation by osteoblasts. Up to date, BMP (bone morphogenic protein, in particular, -2, -4, -7) or PTH (parathyroid hormone) has been known as a bone formation accelerant, and PTH is reported to have an activity of enhancing bone formation in vivo by intermittent systemic administration. Although there is no report saying BMP can enhance bone formation by systemic administration, statin, a low molecular substance inducing BMP expression, has been known to enhance bone formation in vivo (Mundy et al., science 286(5446): 1946-1949, 1999), indicating that the study on signal transduction system of BMP might lead to the development of a low molecular substance inducing bone formation by promoting the production and the activity of a bone formation factor.

There is an eye-opening progress in study on Runx2 gene. Runx family contains three members of Runx1, Runx2 and Runx3, which have been known to play a key role in normal development. According to recent reports, Runx2 (Runx Domain transcription factor) is deeply involved in bone differentiation and has been confirmed to be an important transcription factor regulating the expressions of early marker alkalinephosphatase (ALP) and late marker osteocalcin (OC) (Ducy, P. et al., Cell 89:747-754, 1997; Mundlos, S. et al., Cell 89:773-779, 1997; Komori, T. et al., Cell 89:755-764, 1997; Otto, F. et al., Cell 89:765-771, 1997). In addition, the expression of Runx2 is regulated by Smads (Lee, K. S., et al., Mol. Cell. Biol. 20:8783-8792, 2000), which are signal transmitters activated by BMP (Avantaggiati, M. L. et al., Cell, 89: 1175-1184, 1997). It has also been reported that smurf1 (Smad ubiquitin regulatory factor 1) is a causing factor of ubiquitin-mediated Runx2 protein degradation and signal transduction and osteoblast differentiation by BMP is suppressed by the over-expression of sumrf1 in osteoblast precursor cells (Zhao, M. et al., J. Biol. Chem. 279:12854-12859, 2004; Zhao, M. et al., J. Biol. Chem. 278:27939-27944, 2003).

Therefore, based on the presumption that disclosure of Runx2 regulating mechanism of BMP enables the screening of an accelerant of bone formation, the present inventors further studied and confirmed that BMP can promote osteoblast differentiation by inhibiting smurf1 mediated-Runx2 degradation by Runx2 acetylation. And the present inventors have completed this invention by confirming that bone formation is enhanced by inhibiting Runx2 degradation by using histone deacetylase (HDAC) inhibitor, an inducer of BMP-mediated Runx2 acetylation.

### DISCLOSURE

### Technical Problem

It is an object of the present invention to provide a method for stabilizing Runx2 by acetylation, so that the method can help the prevention and the treatment of bone disease such as osteoporosis, osteogenesis imperfecta, periodontal disease and fracture, by promoting bone formation.

### Technical Solution

To achieve the above object, the present invention provides a method to enhance Runx2 activity by Runx2 acetylation.

The present invention also provides a method to enhance bone formation by Runx2 acetylation.

The present invention further provides a Runx2 acetylase (Runx2 acetylation inducer) containing histone deacetylase inhibitor which promotes the function of BMP (Bone Morphogenetic Protein).

The present invention also provides a composition for the prevention and the treatment of bone disease containing the Runx2 acetylase as an effective ingredient.

Hereinafter, the present invention is described in detail.

The present invention provides a method to enhance Runx2 activity by Runx2 acetylation.

Runx2 is a key regulator of osteogenesis which plays an important role in regulating the expression of bone-specific genes. Runx2 is under the control of various signaling pathways including bone morphogenetic protein (BMP)-mediated signaling pathway. BMP mediates the binding of Smad to Runx2, resulting in the enhancement of bone growth and bone formation.

Runx2 is phosphorylated and activated by MAPK (mitogen-activated protein kinase) pathway which is stimulated by a bone growth factor such as ECM (extracellular matrix) or BMP and a hormone like PTH (parathyroid hormone).

Protein ubiquitination occurring at lysine residues plays an important role in substrate specificity and degradation of a protein by proteosome and Runx2 is degraded by Smurf1 (Smad ubiquitination regulatory factor 1)-mediated ubiquitination. Once Smurf1 is over-expressed, Runx2 is degraded, by which signaling by BMP-2 is intercepted and differentiation of osteoblasts is suppressed.

The concrete molecular mechanism regulating Runx2 protein has not been clearly explained, yet the present inventors found out the fact that the signaling involved in the regulation of Runx2 is associated with Runx2 acetylation. The present inventors also confirmed that Runx2 is a major target of BMP signaling pathway and BMP regulates the transcription level of Runx2 (Lee K. S et al. Mol. Cell. Biol. 20:8783-879, 2000), and thus further investigated if BMP would be involved in post-translational level of Runx2. Particularly, pluripotent stem cells were transfected with myc-labeled Runx2 in the presence or absence of BMP-2, followed by Western blotting using anti-myc antibody. As a result, the level of Runx2 protein was much higher in BMP-2 treated cells (FIG. 1). From the result, it was confirmed that BMP-2 regulates post-translational level of Runx2. To verify the result, HA-labeled Runx2 was co-expressed with increasing the level of BMP receptor 1 (BMPR1). As a result, the expression of Runx2 was increased with the proportion to the level of BMPR 1 (FIG. 2) and the half-life of Runx2 was 12 hours in the presence of BMPR1 but only 2 hours in the absence of BMPR1 (FIG. 3). The above results indicate that the up-regulation of Runx2 by BMP signaling pathway is attributed to the increase of the half-life of Runx2.

RT-PCR was performed to investigate if the level of Runx2 would be increased by BMP mediated transcription. As a result, the level of endogenous Runx2 mRNA was increased while the level of exogenous Runx2 was not changed (FIG. 4), suggesting that the exogenous Runx2 accumulation by BMP-2 is not related to transcription but attributed to the stability of protein. The present inventors confirmed that Runx2 is degraded through ubiquitine-proteosome pathway and Runx2 acetylation induced by BMP-2 contributes to stabilization of Runx2. More precisely, C2C12 cells were transformed with myc-labeled Runx2 in the presence or absence of BMP-2, followed by Western blotting with anti-myc antibody after immunoprecipitation with anti-acetyl-lysine antibody, resulting in the confirmation of Runx2 acetylation (Runx2 acetylation occurs at lysine residues). As a result, a band was detected in Western blot in the presence of BMP-2 (FIG. 5), suggesting that Runx2 acetylation is induced by BMP-2.

The p300 protein functions primarily as a co-activator of transcription for a number of nuclear proteins including E2F1, p53 Smads, BRCA1 and Runx2. p300 functions as a HAT (Histone Acetyl Transferase) and acetylation of specific lysine residues on histone tails is believed to neutralize the negative charge and to generate a more accessible chromatin structure for transcription factors. p300 is also capable of acetylating a number of non-histone proteins. For example, E2F1 is acetylated by p300, resulting in enhancement of DNA-binding capacity and increase of the half-life of protein. The acetylation of E2F1 is reversed by histone deacetylase-1 (HDAC-1) and the increased half-life through the reverse acetylation protects the protein from ubiquitination since acetylation and ubiquitination occur at lysine residues. The present inventors investigated whether or not Runx2 was acetylated by p300 since p300 was bound to SMAD and Runx2 (Kitabayashi I. et al. EMBO J. 17:2994-3004, 1998; Shen, X., et al., Mol. Biol. Cell 9:3309-3319, 1998), and acetylated Runx1 and Runx3 (Gronroos et al. Mol. Cell. 10:483-493, 2002; Jin Y. H. et al. J. Biol. Chem. 279:29409-2941, 2004; Kitabayashi I. et al. EMBO J. 17:2994-3004, 1998). More precisely, p300 was treated instead of BMP-2 by the same manner as described above. As a result, a band was detected in Western blot in the presence of p300, indicating that Runx2 was acetylated by p300 and p300-mediated Runx2 acetylation was increased by BMP (FIG. 5). Similarly, BMPR1, only or together with p300, increased Runx2 acetylation, resulting in the accumulation of the protein (FIG. 6). The above results are consistent with the other test results that the level of a protein increases with the increase of Runx2 acetylation and the level of p300 (FIG. 7). Therefore, it was confirmed that Runx2 acetylation induced by p300 in BMP signaling pathway results in the accumulation of Runx2 protein.

Runx2 contains 10 lysine residues (FIG. 8), 7 lysines in the middle and two other residues at C-terminal. To identify which lysine residue in Runx2 would be acetylated, mutant Runx2-KR, a deletion form of Runx2, was co-expressed with p300, followed by immunoprecipitation with anti-acetyl-lysine antibody. As a result, C-terminal was much more acetylated than other lysine residues (FIG. 9). Particularly, in experiment using a mutant in which lysine was replaced with arginine, it was proved that lysines at 225, 230, 350 and 351 residues of amino acid sequence of Runx2 represented by SEQ. ID. No 1 were the major targets of acetylation by p300 (FIG. 10). And the half-life of Runx2 was approximately 2 hours, but when it was co-expressed with p300, Runx2 became very stable. In the meantime, deletion forms of Runx2-KR 225, 230, 350 and 351, in which lysines at 225, 230, 350 and 351 residues were deleted, were very stable even without p300 (FIG. 11). The above results indicate that acetylation at lysine residues can prevent Runx2 from degradation while Runx2 is sensitively degraded if lysines are not mutated.

Smurf1 is an ubiquitin ligase of Runx2, which recognizes Runx2 to degrade the protein by proteosome (Zhao, M. et al., J. Biol. Chem. 279: 12584-12589, 2004; Zhao, M. et al., J. Biol. Chem. 278: 27939-27944, 2003). Thus, over-expression of Smurf1 accelerates Runx2 degradation (FIG. 12), but co-expression with p300 nullifies Smurf1-mediated Runx2 degradation. In the meantime, deletion forms of Runx2, Runx2-KR 225, 230, 350 and 351, were protected from Smurf1-mediated degradation. Based on the idea that PPxY of Runx2 is reacted with WW domain of Smurf1 (Otte, L. et al., Protein Sci. 12:491-500, 2003) (FIG. 8), the present inventors performed experiments with a mutant of Runx2 having deletion of PPxY, Runx2-del-4,5-418, and another mutant, Runx2-P415R, in which proline at 415 residue was replaced with arginine. As a result, Runx2-del-415-418 and Runx2-P415R were confirmed to be very stable, suggesting that they were protected from Smurf1-mediated degradation (the half-lives of the mutants were more than 24 hours) (FIG. 11). From the above results, it was confirmed that lysines at 225, 230, 350 and 351 residues are the targets of p300-mediated acetylation and Smurf1-mediated ubiquitination. That is, Runx2 acetylation results in non-recognition of lysine residues of Runx2, inhibiting Smurf1-mediated Runx2 degradation (FIG. 12).

The level of Runx2 acetylation is controlled by a dynamic equilibrium of acetylation and deacetylation. That is, when deacetylation is inhibited, Runx2 acetylation is increased. Thus, Runx2 was treated with histone diacetylase (HDAC) inhibitor, which induces deacetylation in most proteins. Particularly, C2C12 cells, transformed with myc-labeled Runx2, were treated with HDAC inhibitor according to the method of Komatsu et al (Cancer Res. 61:4459-4466, 2001). As a result, Runx2 acetylation in C2C12 cells treated with HDAC inhibitor was increased (FIG. 13). The result indicates that Runx2 acetylation/deacetylation is a reversible reaction and HDAC functions as a physiological effector on Runx2 deacetylation. In addition, from the experiment with TβRE luciferase reporter (TβRE-luc) plasmid, in which TGF-β response element inducing transcription by binding with Runx2 was inserted, was confirmed that inhibition of HDAC promotes the activity of the reporter gene. The results indicate that inhibition of HDAC increases transcription activity of Runx2 (FIG. 16).

To confirm Runx2 deacetylation by histone deacetylase, Runx2 and numbers of histone deacetylases were co-introduced. As shown in FIG. 14, histone deacetylases 4 (HDAC4) and 5 (HDAC5) were bound to Runx2, and thus, as shown in FIG. 15, histone deacetylases 4 and 5 deacetylate Runx2.

To investigate the effect of Runx2 acetylation on transcription activity of Runx2, TGF-β response element luciferase (TβRE-Luc) reporter plasmid and Runx2 expression plasmid were co-expressed in 293 cells, followed by treatment with HDAC inhibitors. Then, luciferase was quantified. As a result, it was confirmed that inhibition of HDAC increased the activity of the reporter gene (FIG. 16). That is, inhibition of Runx2 deacetylation increases Runx2 acetylation, which promotes transcriptional activity of Runx2.

To confirm the above result that the increase of transcriptional activity of Runx2 is attributed to the increase of protein level or protein acetylation, the transcriptional activities of Runx2 and Runx2-KR mutant were measured in the absence of p300. As a result, the transcriptional activity of Runx2 was decreased with the mutation of lysine residues targeted by p300 (FIG. 17) and Runx2 activity was not increased at all by p300. The above results indicate that Runx2 acetylation is necessary not only for stabilization of Runx2 but also for transcriptional activity of Runx2.

The present invention also provides a method to enhance bone formation by increasing BMP activity through Runx2 acetylation.

C2C12 pluripotent cells were treated with HDAC inhibitor, followed by alkaline phosphatase activity staining. As a result, the increase of the activity of alkaline phosphatase, an osteoblast marker, was observed (FIG. 18). Runx2 (−/−) cell line, H1-127-21-2, (Lee et al., 2000, Mol. Cell. Biol., Vol. 20, 8783-879) was transfected with luciferase reporter plasmid harboring a promoter of osteocalcin (OC) gene, a marker of late osteoblast differentiation, and was treated with HDAC inhibitor and/or Runx2 expression plasmid. As a result, the activity of OC promoter was also increased by HDAC inhibitor when Runx2 was transfected (FIG. 19). RT-PCR was performed to investigate the expression of OC gene itself in H1-127-21-2 cell line. As a result, the expression level of OC mRNA was increased with the treatment of HDAC inhibitor when Runx2 was transfected, which was consistent with the above result of the increase of OC promoter activity (FIG. 20, 21). HDAC inhibitor was injected hypodermically to a mouse, followed by Hematoxylin and Eosin staining to investigate in vivo bone formation inducing effect. As shown in FIG. 22, HDAC inhibitor was confirmed to induce in vivo bone tissue formation.

Accordingly, the inhibition of HDAC results in the increase of Runx2 acetylation and Runx2 transcriptional activity, which leads to the induction of bone formation.

The present invention further provides a composition for the prevention and the treatment of bone disease containing Runx2 acetylase as an effective ingredient.

It is definitely understood to those in the art that any substance that is able to promote Runx2 acetylation can be included in the present invention and among these substances, deacetylase inhibitor is preferably used.

Runx2 acetylation is mediated by BMP (Bone Morphogenetic Protein) pathway, so BMP or BMP biosynthsis inducer can have the effect of increasing Runx2 acetylation as well (FIG. 5). Besides, Runx2 is controlled by a dynamic equilibrium of acetylation and deacetylation, induced by p300 (acetyl transferase) and HDAC (histone deacetylase), respectively. Thus, Runx2 acetylation is not only stimulated by p300 and BMP but also increased by inhibiting deacetylation by HDAC inhibitor. Co-treatment of BMP or BMP production inducer with HDAC inhibitor accelerates Runx2 acetylation. That is, inhibition of HDAC increases BMP mediated acetylation by inhibiting deacetylation, which means co-treatment of BMP with HDAC inhibitor has synergistic effect on Runx2 acetylation than the single treatment of HDAC inhibitor or BMP. In an example of the present invention, the treatment of HDAC decreased p300-mediated Runx2 acetylation. It has been known that statin compounds such as Lovastatin, Simvastatin, and Compactin, etc, could induce BMP production but not always limited thereto.

Inhibition of HDAC resulted in the increase of the activity of alkaline phosphatase, a marker of early osteoblast differentiation (FIG. 18). From reporter assay with a promoter of osteocalcin gene and RT-PCR to detect the expression of osteocalcin gene, it was also confirmed that inhibition of HDAC increased the activity of the osteocalcin promoter and the expression of the gene (FIG. 19 and FIG. 20). In addition, inhibition of HDAC in a mouse resulted in the promotion of bone formation (FIG. 22).

Therefore, a composition containing the above components as effective ingredients can be effectively used for the prevention and the treatment of bone disease.

The present invention also provides a composition for the prevention and the treatment of bone disease which is characterized by synergistic effect created by co-treatment of HDAC inhibitor with BMP or BMP production inducer.

As described hereinbefore, Runx2 acetylation is mediated by BMP (Bone Morphogenetic Protein) pathway and is controlled by a dynamic equilibrium of acetylation and deacetylation by HDAC (histone deacetylase). So, co-treatment of HDAC inhibitor with BMP or BMP production inducer accelerates Runx2 acetylation. Inhibition of HDAC suppresses deacetylation, indicating that the co-treatment of BMP or BMP production inducer and deacetylase inhibitor increases Runx2 acetylation more than single treatment of HDAC inhibitor. Statins can be used as a BMP production inducer.

The present invention additionally provides a composition for the prevention and the treatment of bone disease having pharmaceutical effect by co-use of osteoclast inhibitor or osteoclast differentiation inhibitor and Runx2 acetylase (HDAC inhibitor).

Bone tissues contain osteoblasts, osteoclasts and osteocytes. Preventive and therapeutic treatments for bone disease such as osteoporosis or periodontal disease depend on inhibition of bone resorption by osteoclasts and enhancement of bone formation by osteoblasts. Osteoclast inhibitor is exemplified by calcitonin and bisphosphonate derivatives, which are now on market, RANKL decoy receptor or RANKL antibody inhibiting osteoclast differentiation, peptide compounds inhibiting the adherence of osteoclasts on bone surface using integrin, c-src tyrosine kinase inhibitor suppressing bone resorption function of osteoclasts, vacuole ATPase inhibitor involved in hydroxyapatite degradation, and cathepsin K inhibitor involved in decomposition of major organisms of bone tissues. Therefore, co-treatment of HDAC inhibitor of the present invention and osteoclast inhibitor increases therapeutic effect on bone disease.

The effective dosage of HDAC inhibitor, in the case of single use, is 10 nM˜10 μM, and is preferred to be 50 nM˜1 μM. In the case of combined treatment with BMP-2, osteoclast inhibitor or statins, the effective dosage of HDAC inhibitor is 10 nM˜500 nM. If the dosage of the inhibitor is less than the above range, preventive or therapeutic effect is not expected to be increased by the combined treatment.

The HDAC inhibitor is characteristically a pharmaceutical composition containing the compound represented by the below formula (1˜14) or its derivatives as an effective ingredient.

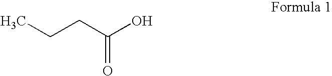

General Formula: C₄H₈O₂

CA index name: butanoic acid (9Cl)

Byname: butyric acid (6Cl, 7Cl, 8Cl); 1-propanecarboxylic acid; ethylacetic acid; honey robber; NSC 8415; propylformic acid; n-butanoic acid; n-butyric acid

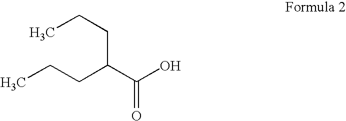

General Formula: C₈H₁₆O₂

CA index name: pentanoic acid, 2-propyl-(9Cl)

Byname: valproic acid, valeric acid, 2-propyl-(6Cl, 7Cl, 8Cl); 2-propylpentanoic acid; 2-propylvaleric acid; 4-heptanecarboxylic acid; 44089; acetic acid, dipropyl-; DPA; depakine; dipropylacetic acid; ergenyl; mylproin; NSC 93819; n-dipropylacetic acid

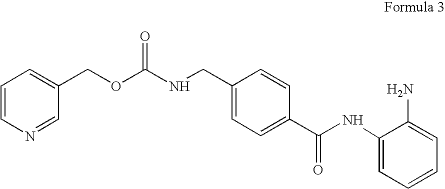

General Formula: C₂₁H₂₀N₄O₃

CA index name: carbamate, [[4-[[(2-aminophenyl)amino]carbonyl]phenyl]methyl]-, 3-pyridinemethyl ester (9Cl)

Byname: MS 27-275; MS 275; MS275-27

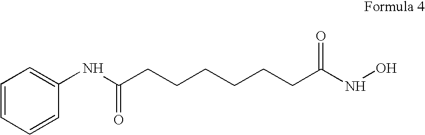

General Formula: C₁₄H₂₀N₂O₃

CA index name: octanediamide, N-hydroxy-N′-phenyl-(9Cl)

Byname: SAHA; Suberoylanilide hydroxamic acid

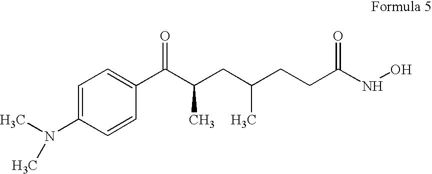

General Formula: C₁₇H₂₂N₂O₃

CA index name: 2,4-heptadieneamide, 7-[4-(dimethylamino)phenyl]-N-hydroxy-4,6-dimethyl-7-oxo-, (2E,4E,6R)-(9Cl)

Byname: 2,4-heptadieneamide, 7-[4-(dimethylamino)phenyl]-N-hydroxy-4,6-dimethyl-7-oxo-, [R-(E,E)]-; (R)-tricostatin A; TSA; tricostatin A

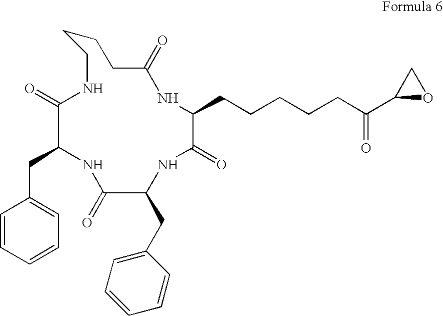

General Formula: C₃₃H₄₀N₄O₆

CA index name: cyclo[(αS,2S)-α-amino-η-oxooxiraneoctanoyl-L-phenylalanyl-L-phenylalanyl-D-prolyl](9Cl)

Byname: cyclo(η-oxo-L-α-aminooxiraneoctanoyl-L-phenylalanyl-L-phenylalanyl-D-prolyl), (S)-; pyrrolo[1,2-a][1,4,7,10]tetraazacyclododecin, cyclic peptide derivative; RF 1023B; trapoxin B

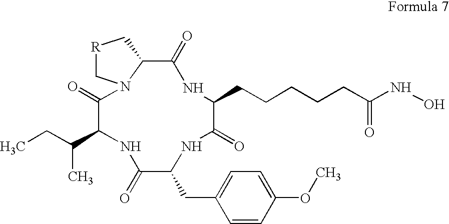

CHAP31 R═CH₂

CHAP50 R═C₂H₄

General Formula: C₃₁H₃₉N₅O₆

CA index name: cyclo[(2S)-2-amino-8-(hydroxyamino)-8-oxooctanoyl-L-phenylalanyl-L-phenylalanylpropyl](9Cl)

CHAP31 cyclo(-L-Asu(NHOH)-D-Tyr(Me)-L-Ile-D-Pro-)

CHAP50 cyclo(-L-Asu(NHOH)-D-Tyr(Me)-L-Ile-D-Pip-)

Byname: CHAP

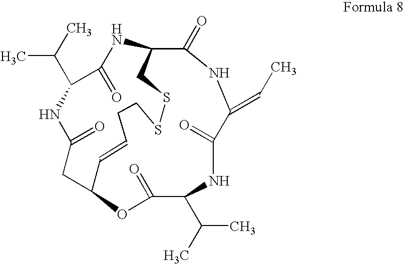

FK228

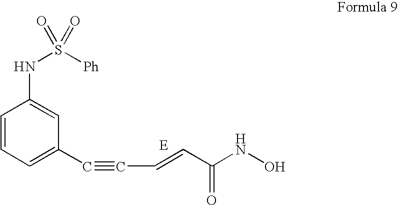

Oxamflatin

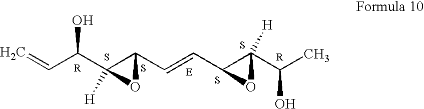

General Formula: C₁₁H₁₆O₄

CA index name: D-threo-D-ido-undeco-1,6-dienitol, 4,5:8,9-dianhydro-1,2,6,7,11-pentadeoxy-(9Cl)

Byname: (−)-depudecin; depudecin

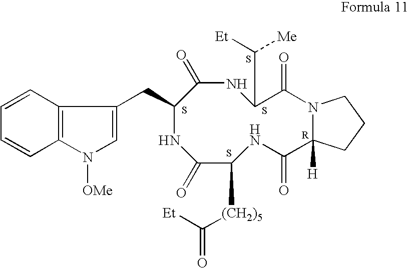

General Formula: C₃₃H₄₇N₅O₆

CA index name: cyclo[(2S)-2-amino-8-oxodecanoyl-1-methoxy-L-triptopyl-L-isoleucyl-D-propyl](9Cl)

Byname: cyclo(8-oxo-L-2-aminodecanoyl-1-methoxy-L-triptopyl-L-isoleucyl-D-propyl); apicidin B; apicidin lb

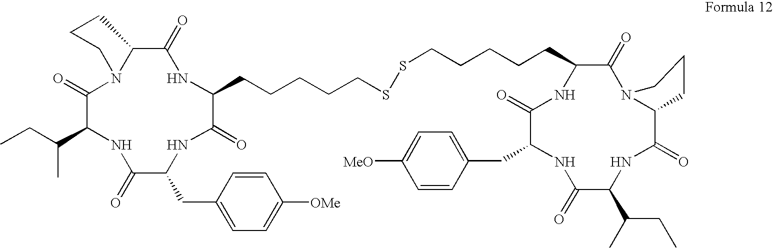

SCOP304

General Formula:

CA index name: cyclo(L-Am7 (−)-D-Tyr(Me)-L-Ile-D-Pro)dimer

byname: compound 7 (Nishino et al., Org. Lett., 5(26), 5079-5082, 2003.)

The derivatives below have similar activities.

cyclo(L-Am7 (−)-D-Tyr (Me)-L-Ile-L-Pro) dimer (derivative a)

cyclo(L-Am7 (−)-D-Tyr (Me)-L-Ile-D-Pip) dimer (derivative b)

cyclo(L-Am7 (−)-D-Tyr (Me)-L-Ile-L-Pip) dimer (derivative c)

cyclo(L-Am6 (−)-D-Tyr (Me)-L-Ile-D-Pro) dimer (derivative d)

cyclo(L-Am8 (−)-D-Tyr(Me)-L-Ile-D-Pro)dimer (derivative e)

cyclo(L-Am9 (−)-D-Tyr (Me)-L-Ile-D-Pro) dimer (derivative f)

The structures of the compounds are described in the following reference (Nishino et al., Org. Lett., 5(26), 5079-5082, 2003.).

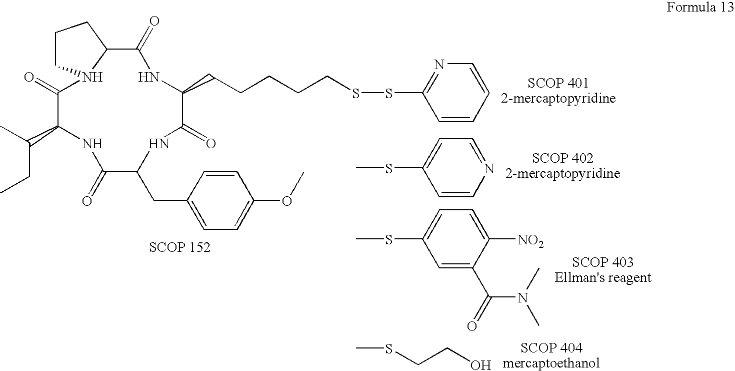

SCOP152 [cyclo(-L-Am7-D-Tyr(Me)-L-Ile-D-Pro-)] and derivatives thereof

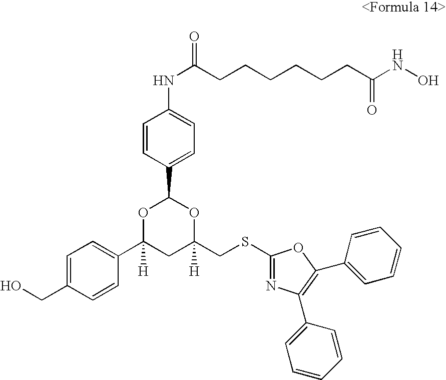

Tubacin

The compounds and their derivatives represented by the above formulas (1˜14) of the present invention stimulate BMP-mediated alkaline phosphatase (APL) activity and the expression of osteocalcin (OC), so that they can be effectively used as a therapeutic agent for bone disease such as osteoporosis or periodontal disease by stimulating bone formation.

The pharmaceutical composition containing HDAC inhibitor as an effective ingredient of the present invention can be administered orally or parenterally and be used in general forms of pharmaceutical formulation.

The composition of the present invention can also include, in addition to the above-mentioned effective ingredients, one or more pharmaceutically acceptable carriers for the administration. Pharmaceutically acceptable carrier can be selected or be prepared by mixing more than one ingredients selected from a group consisting of saline, sterilized water, Ringer's solution, buffered saline, dextrose solution, maltodextrose solution, glycerol and ethanol. Other general additives such as anti-oxidative agent, buffer solution, bacteriostatic agent, etc, can be added. In order to prepare injectable solutions, pills, capsules, granules or tablets, diluents, dispersing agents, surfactants, binders and lubricants can be additionally added. The composition of the present invention can further be prepared in suitable forms for each disease or according to ingredients by following a method represented in Remington's Pharmaceutical Science (the newest edition), Mack Publishing Company, Easton Pa.

The composition of the present invention can be administered orally or parenterally (for example, intravenous, hypodermic, local or peritoneal injection). The effective dosage of the composition can be determined according to weight, age, gender, health condition, diet, administration frequency, administration method, excretion and severity of a disease. The single dosage of HDAC inhibitor is 0.1˜100 mg/kg, and preferably 1˜10 mg/kg. Administration frequency is once a week or preferably a few times a week.

The composition of the present invention can be administered singly or treated with surgical operation, hormone therapy, chemotherapy and biological reaction regulator, to prevent and treat bone disease such as osteoporosis or periodontal disease

### DESCRIPTION OF DRAWINGS

The application of the preferred embodiments of the present invention is best understood with reference to the accompanying drawings, wherein:

FIG. 1 is a photograph showing the result of Western blot analysis measuring the intracellular level of Runx2 increased by BMP-2

FIG. 2 is a photograph showing the result of Western blot analysis measuring the level of Runx2 increased by BMP receptor (BMPR1),

FIG. 3 is a photograph showing the result of Western blot analysis, which is the reducing speed of Runx2 level in the presence or absence of BMPR1 in cells treated with cycloheximide (CHX),

FIG. 4 is a photograph showing the result of semi-quantitative RT-PCR, in which the comparison of endogenous and exogenous Runx2 expressions in cells between transformed with p300 or treated with BMP-2 is made,

FIG. 5 is a photograph showing the result of Western blot analysis measuring the levels of Runx2 and acetylation thereof in cells in which myc-labeled Runx2 and p300 were expressed and BMP was treated,

FIG. 6 is a photograph showing the result of Western blot analysis measuring the levels of Runx2 and acetylation thereof in cells in which myc-labeled Runx2 was expressed in combination with BMPR1 and p300,

FIG. 7 is a photograph showing the result of Western blot analysis measuring the levels of Runx2 and acetylation thereof in cells transformed with a fixed amount of myc-labeled Runx2 and increasing amount of p300,

FIG. 8 is a schematic diagram showing lysine residues of Runx2,

FIG. 9 is a photograph showing the result of Western blot analysis investigating Runx2 acetylation after transfecting cells with p300 and myc-labeled total length or fragmented Runx2,

FIG. 10 is a photograph showing the result of Western blot analysis investigating Runx2 acetylation after transfecting cells with p300 and various myc-labeled Runx2 lysine mutants,

FIG. 11 is a photograph showing the result of Western blot analysis, which is the reducing speed of Runx2 level detected after transfecting cells with myc-labeled Runx2 and various Runx2 mutants in the presence of cycloheximide (CHX),

FIG. 12 is a photograph showing that co-expression of myc-labeled Runx2, numbers of Runx2 mutants and Smurf1 was induced in cells with increasing the contents of them and then the level of Runx2 was measured in the presence or absence of p300,

FIG. 13 is a photograph showing the level of Runx2 and its acetylation in cells treated with HDAC inhibitor,

FIG. 14 is a photograph showing the result of immunoprecipitation investigating interaction between HA-labeled Runx2 and myc-labeled HDAC4 or HDAC5, which were co-expressed in cells,

FIG. 15 is a photograph showing the level of Runx2 and its acetylation in cells in which myc-labeled Runx2 was co-expressed with HDAC4 or HDAC5 and p300,

FIG. 16 is a graph showing the result of luciferase assay performed in the presence or absence of HDAC inhibitor and Runx2. pGL3-TβRE-luc was used as a reporter plasmid,

FIG. 17 is a graph showing the result of luciferase assay, indicating that the gene expression promoting activity of Runx2 was nullified by the replacement of lysine residue with arginine,

FIG. 18 is a photograph showing the result of alkaline phosphatase (APL), an osteoblast marker, assay performed with increasing the concentration of HDAC in the presence of low concentration of BMP-2 (30 ng/ml),

FIG. 19 is a graph showing the result of luciferase assay performed after transfecting cells respectively with wild type OSE and mutant OSE in the presence or absence of Runx2 and HDAC inhibitor SCOP304,

FIG. 20 is a photograph showing the expression level of osteocalcin in cells transformed with Runx2 and treated with HDAC inhibitor,

FIG. 21 is a photograph showing the expression level of osteocalcin in cells transfected with Runx2, Dlx5 or Osx expression plasmid and treated with HDAC inhibitor,

FIG. 22 is a set of photomicrographs showing the enhancement of bone formation in vivo by HDAC inhibitor.

### BEST MODE

Practical and presently preferred embodiments of the present invention are illustrative as shown in the following Examples.

However, it will be appreciated that those skilled in the art, on consideration of this disclosure, may make modifications and improvements within the concept and scope of the present invention.

### Example 1

### Cell Culture and Plasmid Construction

### <1-1> Cell Culture

All cell culture media and antibiotics were from Invitrogen. C2C12 (ATCC No. CRL-1772) and 293 cells were maintained in DMEM with 10% fetal bovine serum (FBS), antibiotics, and anti-mycotics, at 37° C. in 5% CO₂. H1-127-21-2 cells were maintained in alpha-MEM containing 10% FBS, penicillin G (100 U/ml), and streptomycin (100 μg/ml).

### <1-2> Plasmids and Antibodies

Full-length Runx2 (SEQ. ID. No 2) labeled with Myc or HA was inserted into a vector to construct CMV promoter-derived mammalian expression vectors pCS4-3Myc and pCS4-3HA (Jin et al., J Biol Chem. Vol. 279, 29409-17, 2004, Ogawa et al., Proc Natl Acad Sci USA. 1993 Vol. 90, 6859-63.). Mutations affecting the Runx2 acetylation site were introduced by PCR (Michaels A. Innis et al., PCR protocols: a guide to method and applications, Qcademic Press, Inc., 1990) and cloned into the pCS4-3Myc vector. The BMP receptor 1 (BMPR1) and HA-p300 expression vectors were obtained from M. Ewen (Dana-Farber Cancer Institute, Harvard Medical School, Boston) (Genes Dev. Vol. 8, 869-84, 1994). The expression plasmids for the myc-HDAC series were cloned into pCS4-3Myc. The luciferase reporter plasmid, pGL3-TbRE, contains two copies of TbRE (originally identified in the immunoglobulin Cα promoter) (Lee, K. S., et al., Mol. Cell. Biol. 20:8783-8792, 2000; pGL3 is product of Promega). The promoter region of rat osteocalcin (−1050 OC-CAT, −1050/+23) was subcloned into the pGL2-basic vector and named pGL21050 OC-luc (Hoffamann et al., J Cell Biochem. 2000, vol. 80, 156-68) (provided from Dr. Lian (Department of Cell Biology, University of Massachusetts Medical Center, Worcester, Mass. 01655, USA)).

The wild type and mutated OSE sites (the Runx2 binding site C of the OC promoter region (−208/+23)) have been described previously (Kim, H. J. et al., J. Biol. Chem. 278: 319-326, 2003). In this invention, antibodies against acetyl-lysine (Cell Signaling Technology), Myc (9E10, Santa Cruz Biotechnology) and HA (12CA5, Roche) were used.

### Example 2

### Reporter Assay and Immunoblotting

### <2-1> Transfection

Transient transfections were performed using the calcium phosphate method for 293 cells or the Lipofectamine Plus reagent (Invitrogen) for the C2C12 cell line and H1-127-21-2 cell line established from Runx2(−/−) mouse calvaria (Lee, K. S., et al., Mol. Cell. Biol. 20:8783-8792, 2000) according to the manufacturer's recommendations.

### <2-2> Reporter Assay

For luciferase assay, cells were plated on a 24-well plate one day before transfection, followed by co-transfection with luciferase reporter plasmid and numbers of Runx2 constructs. 36 hours after transfection, cells were recovered and luciferase and β-galactosidase activities were measured in cell lysate by using Luciferase Reporter Assay Kit (Promega) using luminometer according to the manufacturer's recommendations. The pCMVβ-Gal (beta-Galactosidase, Clontech Laboratory) plasmid was included as an internal control to determine the efficiency of transfection.

### Example 3

### Immunoprecipitation and Immunoblotting

Following transfection, the C2C12 and 293 cells were lysed in ice-cold cell lysis buffer (25 mM HEPES (pH 7.5), 150 mM NaCl, 1% NP-40, 0.25% Na deoxycholate, 10% glycerol, 25 mM NaF, 1 mM EDTA, 1 mM Na₃VO₄, 250 μM phenylmethylsulfonyl fluoride, 10 μg/ml leupeptin, 10 μg/ml aprotinin) and cleared by centrifugation. The resulting supernatants were immunoprecipitated with the appropriate primary and secondary antibodies, and protein A- or protein G-Sepharose beads (Amersham) for 4 hours. All incubations were conducted at 4° C. The Sepharose beads were washed extensively and bound proteins were separated using SDS-PAGE, and then transferred to PVDF membranes. The membranes were incubated with the appropriate antibodies, washed, and then incubated with horseradish peroxidase-coupled secondary antibodies. After washing, the reactive proteins were visualized with an enhanced chemiluminescence (ECL) reagent (Amersham Biosciences).

For HDAC inhibitor treatments, both C2C12 and 293 cells were cultured in fresh medium for 1 day following transfection, and then treated with the inhibitors (Tricostatin A (TSA), CHAP and SCOP) for 16 h, at the specified concentrations according to the methods of Komatsu et al (Cancer Res. 61:4459-4466, 2001) and Nishino et al (Nishino N. et al., Org. Lett. 5: 5079-5082, 2003).

### Example 4

### BMP-2 Mediated Runx2 Acetylation

### <4-1> Increase of Runx2 Stability Attributed to BMP-2

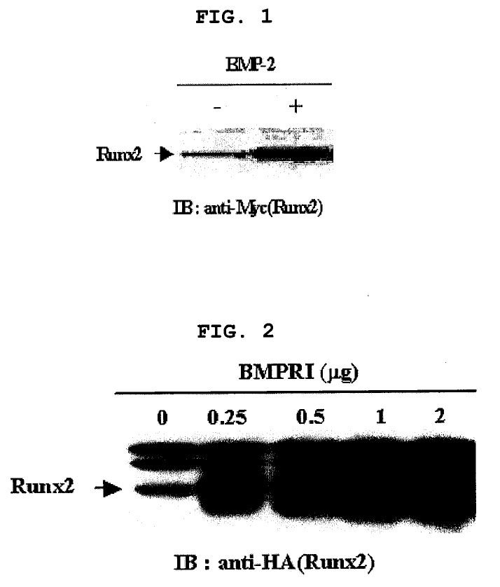

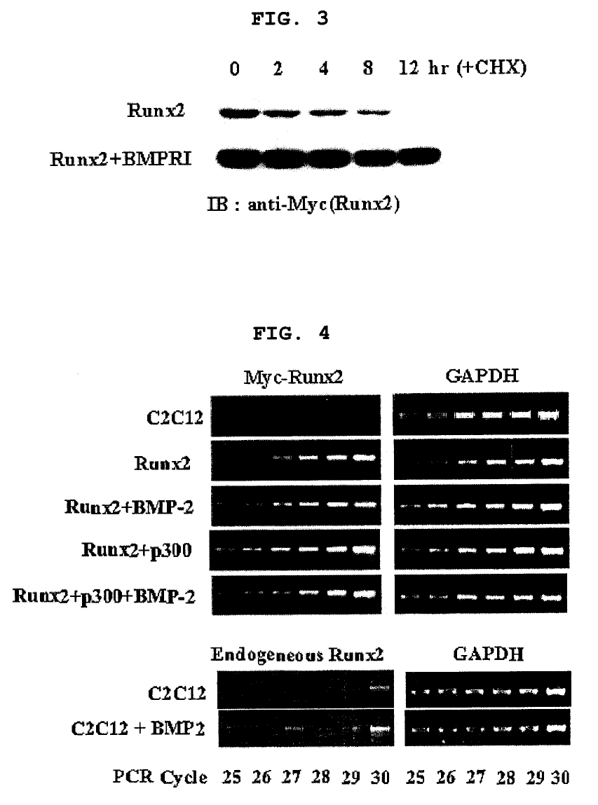

After confirming that Runx2 is a major target of BMP-2 signaling pathway and BMP-2 regulates transcription level of Runx2 (Lee K. S et al. Mol. Cell. Biol. 20:8783-879, 2000), the present inventors investigated whether BMP-2 regulates post-transcription level of Runx2 as well. Particularly, C2C12 pluripotent mesenchymal precursor cells were transfected with myc-tagged Runx2 in the presence or absence of 300 ng/l of BMP-2, followed by Western blotting using anti-myc antibody. As a result, the level of Runx2 protein was much higher in BMP-2 treated cells (FIG. 1). The result was confirmed by co-expression of HA-tagged Runx2 and increasing amount of BMP receptor 1 (BMPR1) of 0, 0.25, 0.5, 1 and 2 μg. That is, the level of Runx2 increases with proportion to the amount of BMPR1 (FIG. 2). 293 cells were transfected with myc-tagged Runx2, to which a protein synthesis inhibitor cycloheximide (CHX) was added by 40 μg/ml, followed by Western blotting using myc antibody in the presence or absence of BMPR1, at the time point of 0, 2, 4, 6, 8 and 12 hour. The half-life of Runx2 was 12 hours in the presence of BMPR1 but only 2 hours in the absence of BMPR1 (FIG. 3). To eliminate the possibility of BMP-2 mediated transcriptional activity of the transfected Runx2 gene, the present inventors measured the level of Runx2 by BMP-2 transcriptional activity using quantitative RT-PCR. Precisely, 293 cells were transfected with p300 or BMP-2, followed by observation on Runx2 expression. RT-PCR was performed to measure the levels of endogenous and exogenous Runx2 levels. To quantify endogenous Runx2 level, primers represented by SEQ. ID. No 3 and No 4 were used for RT-PCR while primers represented by SEQ. ID. No 5 and No 6 were used to measure exogenous level. DNA polymerase (Taq polymerase, Takara) was used for PCR. PCR was performed as follows; predenaturation at 95° C. for 5 minutes, denaturation at 95° C. for 30 seconds, annealing at 55° C. for 1 minute, polymerization at 72° C. for 1 minute, 30 cycles from denaturation to polymerization. Following quantitative RT-PCR, PCR products were collected (1/20 of reaction mixture) from several reaction cycles (from 25 to 30), and fragments were separated using agarose gel electrophoresis.

As a result, endogenous Runx2 mRNA level was increased but exogenous Runx2 mRNA level was not changed (FIG. 4). The results indicate that the accumulation of exogenous Runx2 protein, in response to BMP-2 signaling, is independent of the increase in transcription, but is associated with increased stability of the protein.

### <4-2> BMP-2 Mediated Runx2 Acetylation

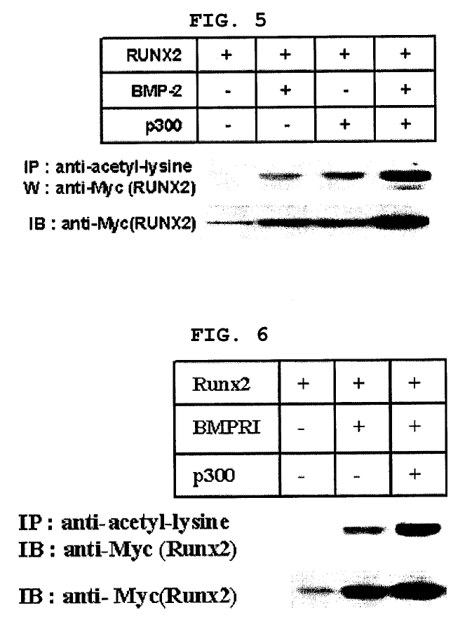

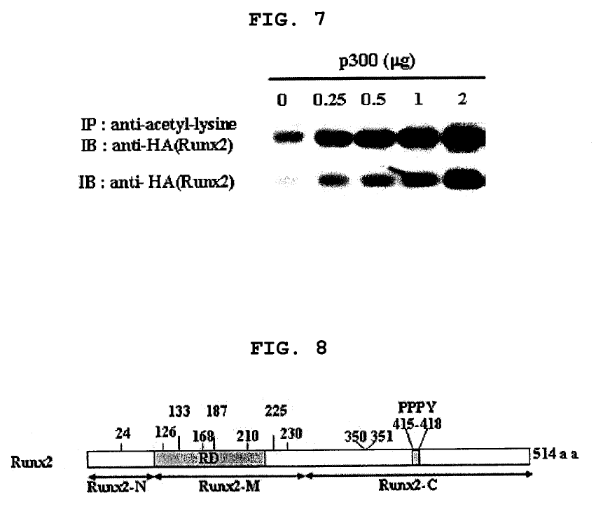

Since Runx2 is degraded via the ubiquitine-proteosome mediated pathway and acetylation can protect proteins from ubiquitination, the present inventors further examined whether BMP-2 induces Runx2 acetylation, thereby stabilizing the Runx2 protein. Particularly, C2C12 cells were transfected with myc-tagged Runx2 in the presence or absence of BMP-2 and p300, followed by immunoprecipitation using anti-acetyl-lysine antibody, leading to Western blot analysis using anti-Myc antibody (Runx2 acetylation occurred at lysine residues). As a result, Runx2 was acetylated by BMP-2 and p300 treatment. A band was detected in the presence of either BMP-2 or p300, and a band was more clearly detected by co-treatment of BMP-2 and p300 (FIG. 5), indicating that p300 mediated Runx2 acetylation is accelerated by BMP-2 treatment.

### Example 5

### p300 Mediated Runx2 Acetylation

Since p300 is bound with SMAD and Runx2 (Kitabayashi I. et al. EMBO J. 17:2994-3004, 1998; Shen, X., et al., Mol. Biol. Cell 9:3309-3319, 1998), and Runx1 and Runx3 are targets of p300 acetyltransferase activity (Gronroos et al. Mol. Cell. 10:483-493, 2002; Jin Y. H. et al. J. Biol. Chem. 279:29409-2941, 2004; Kitabayashi I. et al. EMBO J. 17:2994-3004, 1998), the present inventors examined whether p300 is associated with BMP-2-mediated Runx2 acetylation. Experiments were performed by the same manner as described in Example 4 except the treatment of p300 and BMPR1. As a result, a band was detected in Western blot in the presence of p300, and a band was more clearly detected in the presence of p300 and BMPR1 together. The results indicate that Runx2 is acetylated by p300 and p300-mediated Runx2 acetylation is accelerated by the treatment of BMPR1 (FIG. 6).

Therefore, it was confirmed that the accumulation of Runx2 protein is dependent of p300-mediated Runx2 acetylation induced by BMP signaling pathway.

### Example 6

### Runx2 Acetylation Site

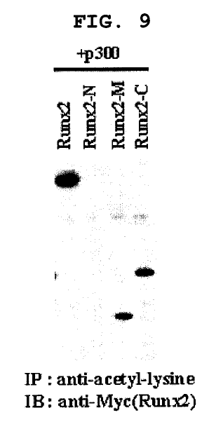

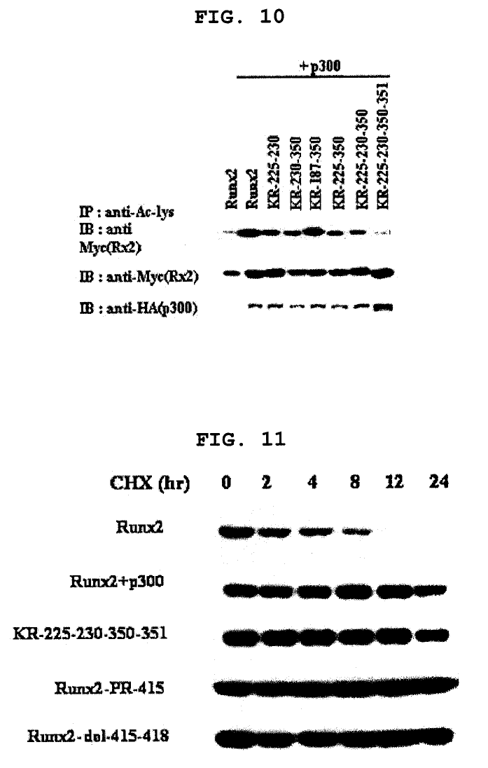

Runx2 contains 10 lysine residues, 7 lysine residues in the middle and 2 lysine residues at C-terminal (FIG. 8). To identify which residues are targets of acetylation, Runx2-KR, a Runx2 deletion mutant, was co-expressed with p300, and cell lysates were immunoprecipitated using an anti-acetyl-lysine antibody. As a result, more acetylation was observed at C-terminal region (FIG. 9). Acetylation in Runx2 mutants, in which lysine is substituted with arginine, was also investigated. As a result, 225^{th}, 230^{th}, 350^{th} and 351^{st} lysine residues of Runx2 amino acid sequence represented by SEQ. ID. No 1 were proved to be major targets of p300-mediated acetylation (FIG. 10).

### Example 7

### Protection of Protein from Smurf1-Mediated Degradation by Runx2 Acetylation

The half-life of Runx2 is approximately 2 hours. When Runx2 is expressed together with p300, it becomes very stable. In the meantime, the Runx2-KR 225, 230, 350 and 351 mutants were very stable even without the treatment of p300 (FIG. 11). The results indicate that Runx2 is sensitively degraded without lysine mutation but is protected from degradation by lysine mutation.

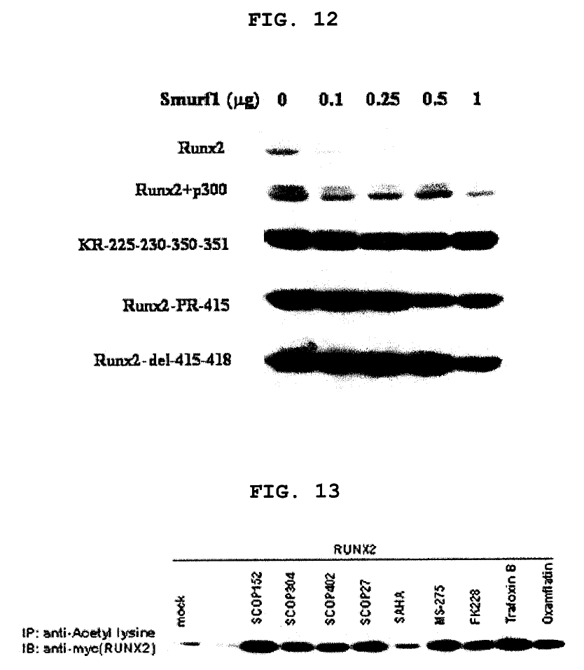

Smurf1 functions as a Runx2 ubiquitine ligase, recognizing and degrading Runx2 by proteosome (Zhao, M. et al., J. Biol. Chem. 279: 12584-12589, 2004; Zhao, M. et al., J. Biol. Chem. 278: 27939-27944, 2003). Thus, over-expression of Smurf 1 accelerates degradation of Runx2 (FIG. 12) but co-expression of Smurf1 and p300 nullifies Smurf1-mediated Runx2 degradation. However, Runx2-KR 225, 230, 350 and 351 mutants are strongly resistant to Smurf1-mediated degradation. Runx2 contains PPxY motif that is required for interaction with the WW domain of Smurf1 (Otte, L. et al., Protein Sci., 12:491-500, 2003) (FIG. 8). Thus, PPxY deleted Runx2-del-415-418 and Runx2-P415R, in which proline at 415^{th} residue was replaced with arginine, were used for experiment (Michaels A. Innis et al., PCR protocols: a guide to method and applications, Qcademic Press, Inc., 1990). As a result, it was confirmed that Runx2-del-415-418 and Runx2-P415R were very stable, which means they were strongly resistant to Smurf1-mediated degradation (the half-lives of them were more than 24 hours) (FIG. 11). Therefore, lysines at 225^{th}, 230^{th}, 350^{th} and 351^{st} residues were confirmed to be major targets of p300-mediated acetylation and Smurf1-mediated ubiquitination.

### Example 8

### HDAC and Runx2 Acetylation

### <8-1> Runx2 Acetylation by HDAC Inhibitor

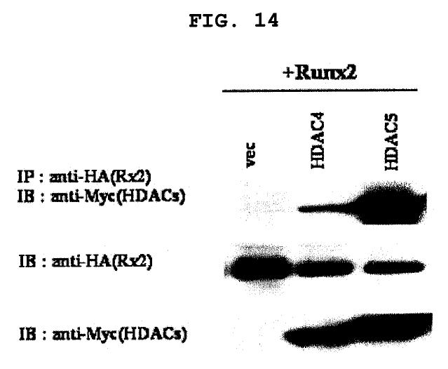

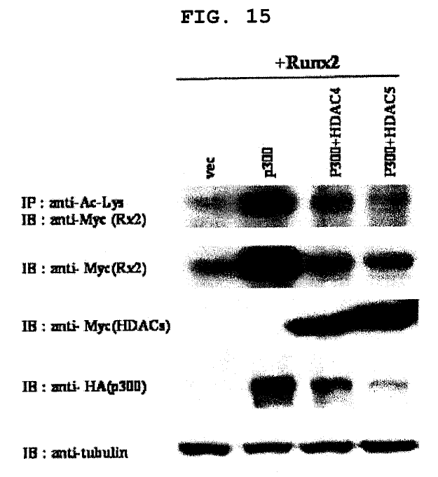

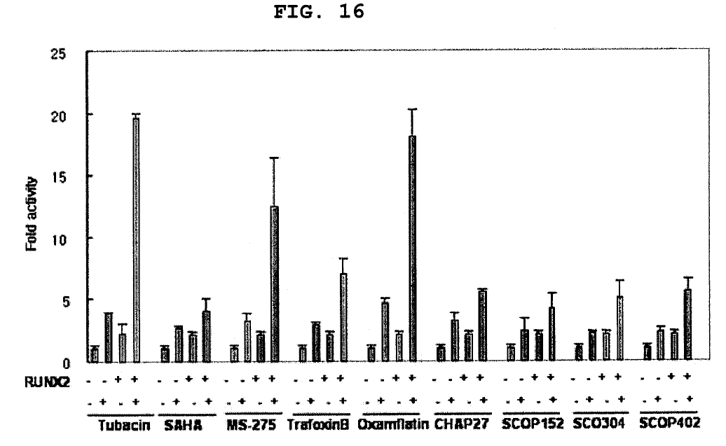

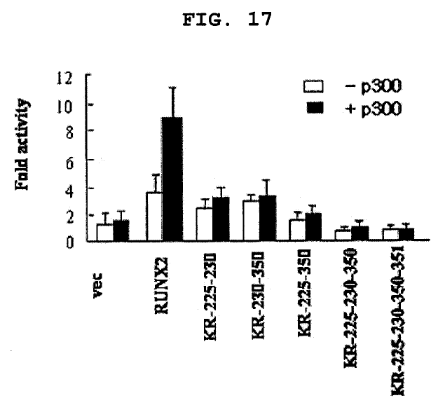

The level of Runx2 acetylation might be controlled by a dynamic equilibrium between acetylation and deacetylation. If then, inhibition of deacetylation results in increase of Runx2 acetylation. In the present invention, inhibitors of histone diacetylase (HDAC) deacetylating most proteins were treated to Runx2. Particularly, C2C12 cells, transfected with myc-tagged Runx2, were treated with HDAC inhibitors such as SCOP304, SCOP152, CHAP27, SAHA, MS-275, FK228, TrafoxinB and Oxamflatin according to the method of Komatsu et al (Cancer Res. 61:4459-4466, 2001). Immunoprecipitation was performed using anti-acetyl-lysine antibody, followed by Western blot analysis using anti-myc antibody. As a result, Runx2 acetylation in C2C12 cells treated with HDAC inhibitors was increased (FIG. 13). Physical interaction between Runx2 and HDAC4 or HDAC5 was confirmed by co-immunoprecipitation (FIG. 14) and HDAC4 or HDAC5 mediated Runx2 deacetylation was also confirmed by Western blot analysis using anti-myc antibody performed after immunoprecipitation using anti-acetyl-lysine antibody in cells transfected with Runx2 and HDAC (FIG. 15).

As described above, the result that Runx2 acetylation is decreased by HDAC and increased by inhibition of deacetylation indicates that Runx2 acetylation/deacetylation is a dynamic process and HDAC may be the physiological effector of Runx2 deacetylation.

### Example 9

### Promotion of Runx2 Transcriptional Activity by HDAC Inhibitor

To examine whether HDAC inhibitors, increasing the level of Runx2 acetylation, could promote the Runx2 transcriptional activity, 293 cells over-expressing Runx2 and pGL3-TβRE-luc reporter were treated with each HDAC inhibitor. The transcriptional activity of Runx2 was evaluated by luciferase report assay, which was performed according to the manufacturer (Promega)'s instructions. As a result, HDAC inhibitors, Tubacin, SAHA, MS-275, Trafoxin B, Oxamflatin, CHAP27, CHAP31, SCOP152, SCOP304 and SCOP402, increased transcriptional activity of Runx2 (FIG. 16). From the result, it was confirmed that HDAC inhibitors not only increase the level of Runx2 acetylation but also increase the Runx2 transcriptional activity.

### Example 10

### Enhancement of Osteoblast Differentiation by HDAC Inhibitor

### <10-1> Activation of Alkaline Phosphatase by HDAC Inhibitor

Runx2 is a key regulator of osteoblast differentiation and a major target of BMP-2 signaling. Thus, the present inventors further examined whether HDAC inhibitors stabilizing Runx2 by increasing Runx2 acetylation could induce osteoblast differentiation as well.

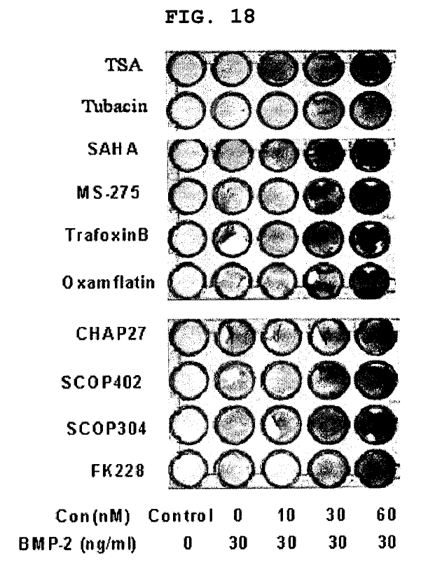

Particularly, C2C12 pluripotent mesenchymal precursor cells were treated with each HDAC inhibitor and then fixed with 3.7% formaldehyde for 10 minutes, to which western blue substrate (Promega) was added to induce color development for 10 minutes. And alkaline phosphatase (ALP) activity, an early marker of osteoblast differentiation was measured by the active staining method (Katagiri et al., J. Cell Biol. Vol. 127, 1755-66, 1994). As a result, the treatment of HDAC inhibitors such as TSA, SAHA, MS-275, FK228, Oxamflatin, CHAP27, SCOP152, SCOP304 and SCOP402 caused increase of ALP activity (FIG. 18).

### <10-2> Enhancement of Osteocalcin Expression by HDAC Inhibitor

In order to confirm the stimulating effect of Runx2 acetylation on osteoblast differentiation, the present inventors examined whether osteoblast differentiation could induce osteocalcin (OC, a late marker of osteoblast differentiation), that is, whether HDAC inhibitors could effect on OC promoter.

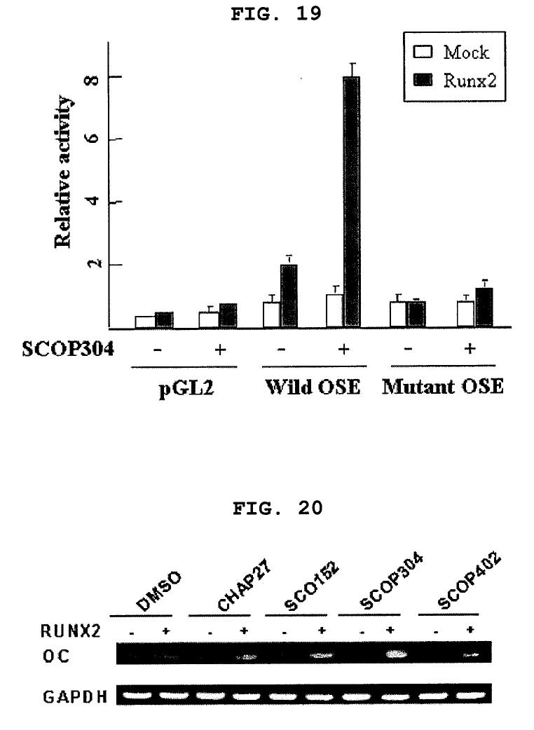

Particularly, pCS4-3Myc-Runx and pGL2-1050 OC-luc were over-expressed in H1-127-21-2 cells which were established from Runx2(−/−) mouse calvaria (Lee et al., 2000, Mol. Cell. Biol., Vol. 20, 8783-879). The cells were treated with HDAC inhibitor SCOP304 according to the conventional treatment method of HDAC inhibitor (Nishino N. et al., Org. Lett. 5: 5079-5082, 2003). Then, luciferase report assay was performed according to the manufacturer's instructions to evaluate OC promoter activity. As a result, HDAC inhibitors activated the OC promoter 2-4 fold over the activation observed for Runx2 alone (FIG. 19). OC promoter activity of Runx2 binding site (OSE) was completely abrogated either by HDAC inhibitors or by Runx2 overexpression (FIG. 19).

RT-PCR was performed to investigate the effect of HDAC inhibitors on OC gene expression. Particularly, RT-PCR was performed to quantify the OC gene expression using primers represented by SEQ. ID. No 7 and No 8. DNA polymerase (Taq polymerase, Takara) was used for PCR. PCR was performed as follows; predenaturation at 95° C. for 5 minutes, denaturation at 95° C. for 30 seconds, annealing at 55° C. for 1 minute, polymerization at 72° C. for 1 minute, 30 cycles from denaturation to polymerization, and final extension at 72° C. for 10 minutes. As a result, OC expression was three-fold increased by HDAC inhibitors in the presence of Runx2. The result was consistent with the result of OC promoter activity analysis (FIG. 20).

### Example 11

### OC Expression Increased Specifically by Runx2 Acetylation

OC expression is regulated by other bone formation transcription factors such as Dix5 (Lee, M. H., et al., J. Cell Biochem. 73: 114-125, 1999) or Osterix (Osx) (Nakashima, K. et al., Cell 108: 17-29, 2002). The present inventors investigated whether Runx2 is a specific target of HDAC inhibitors for activation of OC promoter using same primers and RT-PCR conditions of Example 10. Runx2(−/−) cells were transfected with Runx2, Dix5 (Lee M. H. et al., J. Biol. Chem. 278, 34387-34394, 2003) and Osterix (Nakashima, K et al., Cell 108: 17-29, 2002) genes using Lipofectamin PLUS REAGENT (Invitrogen) according to the manufacturer's instructions, and 6 hours later, the cells were treated with HDAC inhibitors. 24 hours later, the level of OC mRNA was measured by RT-PCR. The primers and reaction conditions hired for RT-PCR amplifying OC mRNA were the same as described in Example <10-2>.

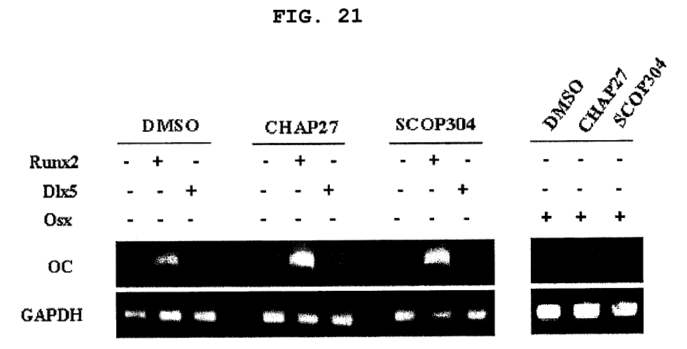

As a result, OC expression in Runx2(−/−) cells not expressing Runx2 was increased by exogenous Runx2 and the addition of HDAC inhibitors enhanced OC expression. In the meantime, the treatment of Dlx5 or Osx slightly increased OC expression but the expression was no more increased even with the addition of HDAC inhibitors (FIG. 21). The results indicate that only Runx2, among three bone formation transcription factors, is a major target of HDAC inhibitors in osteoblast differentiation.

### Example 12

### Enhancement of Bone Formation by HDAC Inhibitor

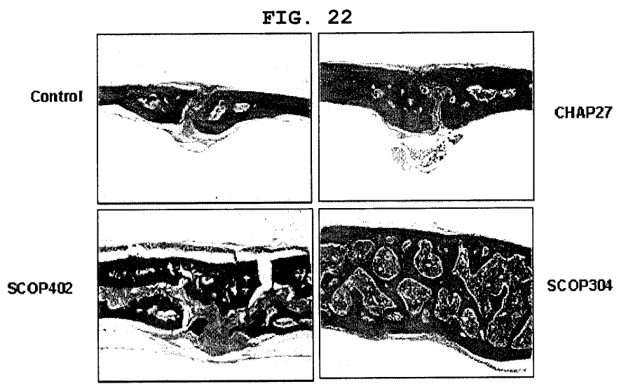

To confirm the effect of HDAC inhibitors on bone formation, the present inventors investigated the effect of CHAP27, SCOP402 and SCOP304 on periosteal bone formation in rodent. Particularly, 10 μl of HDAC inhibitors (60 μM) were hypodermically injected into mice calvarias (Zhao et al., J. Biol. Chem., 279, 12854-12859, 2004). Two weeks later, the mice were sacrificed, followed by hematoxyline & eosine histostaining (Cell Biology. Second edition, Edited by Julio E. Celis. Published by Academic Press, 1994). From the observation, it was confirmed that CHAP27 and SCOP304 remarkably enhanced bone formation (FIG. 22).

The above results indicate that HDAC inhibitors enhance bone formation in vivo and Runx2 acetylation/deacetylation is an important mechanism to regulate the function of osteoblasts.

### INDUSTRIAL APPLICABILITY

As described hereinbefore, the method enhancing Runx2 activity by Runx2 acetylation can greatly contribute to the treatment of bone disease including osteoporosis, osteogenesis imperfecta, periodontal disease and fracture by protecting Runx2 from degradation, thereby stimulating bone formation.

### Sequence List Text

SEQ. ID. No 1 is an amino acid sequence of Runx2,

SEQ. ID. No 2 is a DNA sequence of Runx2,

SEQ. ID. No 3 and No 4 are respectively forward and reverse primers used for the amplification of endogenous Runx2,

SEQ. ID. No 5 and No 6 are respectively forward and reverse primers used for the amplification of exogenous Runx2,

SEQ. ID. No 7 and No 8 are respectively forward and reverse primers used for the RT-PCR of OC gene.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method to enhance Runx2 activity by Runx2 acetylation.

2. The method to enhance Runx2 activity as set forth in claim 1, wherein the Runx2 activity is transcriptional activity.

3. The method to enhance Runx2 activity as set forth in claim 1, wherein the Runx2 activity is enhanced by Runx2 stabilization through acetylation of the Runx2.

4. The method to enhance Runx2 activity as set forth in claim 2, wherein the Runx2 stabilization is enhanced by increasing half-life of the acetylated Runx2.

5. The method to enhance Runx2 activity as set forth in claim 1, wherein the Runx2 activity is regulated by BMP.

6. The method to enhance Runx2 activity as set forth in claim 1, wherein the Runx2 activity is regulated by p300.

7. The method to enhance Runx2 activity as set forth in claim 1, wherein the Runx2 activity is regulated by Smurf1-mediated ubiquitination.

8. The method to enhance Runx2 activity as set forth in claim 5, claim 6 or claim 7, wherein the Runx2 acetylation sites are lysines of Runx2 amino acid sequence represented by SEQ. ID. No 1.

9. The method to enhance Runx2 activity as set forth in claim 1, wherein the Runx2 activity is enhanced by inhibition of deacetylation of acetylated Runx2.

10. The method to enhance Runx2 activity as set forth in claim 9, wherein the deacetylation is accomplished by histone deacetylases.

11. The method to enhance Runx2 activity as set forth in claim 1, wherein the inhibition of deacetylation is accomplished by histone deacetylase inhibitor selected from a group consisting of butanoic acid, pentanoic acid, carbamate, octanediamide, 2,4-heptadieneamide, cyclo[(αS,2S)-α-amino-η-oxooxiraneoctanoyl-L-phenylalanyl-L-phenylalanyl-D-prolyl](9Cl), cyclo[(2s)-2-amino-8-(hydroxyamino)-8-oxooctanoyl-L-phenylalanyl-L-phenylalanylpropyl](9Cl), oxamflatin, D-threo-D-ido-undeco-1,6-dienitol, 4,5,8,9-dianhydro-1,2,6,7,11-pentadeoxy-(9Cl), cyclo[(2S)-2-amino-8-oxodecanoyl-1-methoxy-L-tryptopyl-L-isoleucyl-D-propyl](9Cl), cyclo(L-Am7(−)-D-Tyr (Me)-L-Ile-D-Pro dimer, cyclo(-L-Am7-D-Tyr(Me)-L-Ile-D-Pro-), tubacin and derivatives thereof.

12. A method to enhance bone formation by Runx2 acetylation.

13. The method to enhance bone formation as set forth in claim 12, wherein the bone formation is enhanced by Runx2 stabilization through acetylation of the Runx2.

14. The method to enhance bone formation as set forth in claim 12, wherein the Runx2 acetylation is regulated by BMP.

15. The method to enhance bone formation as set forth in claim 12, wherein the Runx2 acetylation is regulated by p300.

16. The method to enhance bone formation as set forth in claim 13, claim 14 or claim 15, wherein the Runx2 acetylation sites are lysines of Runx2 amino acid sequence represented by SEQ. ID. No 1.

17. The method to enhance bone formation as set forth in claim 12, wherein the bone formation is enhanced by inhibition of deacetylation of acetylated Runx2.

18. The method to enhance bone formation as set forth in claim 17, wherein the deacetylation is accomplished by histone deacetylase.

19. The method to enhance bone formation as set forth in claim 18, wherein the inhibition of deacetylation is accomplished by histone deacetylase inhibitor selected from a group consisting of butanoic acid, pentanoic acid, carbamate, octanediamide, 2,4-heptadieneamide, cyclo[(αS,2S)-α-amino-η-oxooxiraneoctanoyl-L-phenylalanyl-L-phenylalanyl-D-prolyl](9Cl), cyclo[(2s)-2-amino-8-(hydroxyamino)-8-oxooctanoyl-L-phenylalanyl-L-phenylalanylpropyl](9Cl), oxamflatin, D-threo-D-ido-undeco-1,6-dienitol, 4,5,8,9-dianhydro-1,2,6,7,11-pentadeoxy-(9Cl), cyclo[(2S)-2-amino-8-oxodecanoyl-1-methoxy-L-tryptopyl-L-isoleucyl-D-propyl] (9Cl), cyclo(L-Am7 (−)-D-Tyr(Me)-L-Ile-D-Pro dimer, cyclo(-L-Am7-D-Tyr(Me)-L-Ile-D-Pro-), tubacin and derivatives thereof.

20. The method to enhance bone formation as set forth in claim 12, wherein the bone formation is enhanced by acceleration of osteoblast differentiation.

21. The method to enhance bone formation as set forth in claim 12, wherein the bone formation is promoted by the increase of expressions of osteoblast marker genes including alkaline phosphatase and or osteocalcin.

22. A composition for the prevention and the treatment of bone disease containing Runx2 acetylase as an effective ingredient.

23. The composition for the prevention and the treatment of bone disease as set forth in claim 22, wherein the Runx2 acetylase is selected from a group consisting of histone deacetylase inhibitors activating BMP-mediated bone formation pathway.

24. The composition for the prevention and the treatment of bone disease as set forth in claim 22, wherein the histone deacetylase inhibitor is selected from a group consisting of butanoic acid, pentanoic acid, carbamate, octanediamide, 2,4-heptadieneamide, cyclo[(αS,2S)-α-amino-η-oxooxiraneoctanoyl-L-phenylalanyl-L-phenylalanyl-D-prolyl] (9Cl), cyclo[(2s)-2-amino-8-(hydroxyamino)-8-oxooctanoyl-L-phenylalanyl-L-phenylalanylpropyl](9Cl), oxamflatin, D-threo-D-ido-undeco-1,6-dienitol, 4,5,8,9-dianhydro-1,2,6,7,11-pentadeoxy-(9Cl), cyclo[(2S)-2-amino-8-oxodecanoyl-1-methoxy-L-tryptopyl-L-isoleucyl-D-propyl](9Cl), cyclo(L-Am7(−)-D-Tyr(Me)-L-Ile-D-Pro dimer, cyclo(-L-Am7-D-Tyr(Me)-L-Ile-D-Pro-), tubacin and derivatives thereof.

25. The composition for the prevention and the treatment of bone disease as set forth in claim 22, which additionally includes osteoclast inhibitor or osteoclast differentiation inhibitor.

26. The composition for the prevention and the treatment of bone disease as set forth in anyone of claim 22˜claim 24, which additionally includes BMP or BMP production inducer to provide synergy effect.

27. The composition for the prevention and the treatment of bone disease as set forth in claim 26, wherein the BMP production inducers are statins.

28. The composition for the prevention and the treatment of bone disease as set forth in claim 27, wherein the statin is selected from a group consisting of lovastatin, simvastatin, compactin and derivatives thereof.

29. The composition for the prevention and the treatment of bone disease as set forth in claim 22, wherein the bone disease is selected from a group consisting of osteoporosis, osteogenesis imperfecta, fracture and periodontal disease.

30. A Runx2 acetylase containing histone deacetylase as an effective ingredient.

31. An accelerant of osteoblast differentiation containing histone deacetylase as an effective ingredient.

32. An accelerant of osteocalcin expression containing histone deacetylase as an effective ingredient.

33. An accelerant of bone formation containing histone deacetylase as an effective ingredient.
